(19) United States
(12) Patent Application Publication
(10) Pub. No.: US 2010/0298244 A1
(43) Pub. Date: Nov. 25, 2010

(54) COMPOSITION FOR THE PREVENTION AND THE TREATMENT OF HELICOBACTER PYLORI INFECTION

(75) Inventors: Jyh-Chin Yang, Taipei City (TW); Chia-Tung Shun, Taipei City (TW); Chiang-Ting Chien, Taipei City (TW); Teh-Hong Wang, Taipei City (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL TAIWAN UNIVERSITY, Taipei City (TW)

(21) Appl. No.: 12/604,635

(22) Filed: Oct. 23, 2009

(30) Foreign Application Priority Data
May 21, 2009 (TW) 098116887

(51) Int. Cl. A61K 31/7004; A61K 31/352

(52) U.S. Cl. 514/23; 514/456

(57) ABSTRACT

The disclosed item is a composition comprising catechins and sialic acid to provide an effective prevention and treatment for Helicobacter Pylori infection. This composition inhibits growth of Helicobacter pylori, and can increase the antioxidant activity of gastric mucosal cells through reducing the production of O₂.⁻, H₂O₂ and NO, and decreasing the expression of inducible nitric oxide synthase (iNOS) in cells. The apoptotic damage of stomach caused by Helicobacter pylori was decreased through up-regulation of autophagy to enhance the defensive ability of gastric epithelial cells toward Helicobacter pylori. Therefore the composition can be used in the treatment or prevention on damages caused by Helicobacter pylori infection.

### BACKGROUND OF THE INVENTION

1. Field of the Invention

The present invention relates to a non-antibiotic composition for use in the treatment or the prevention of Helicobacter pylori infection, especially a composition comprising catechins and sialic acid for use in the treatment or the prevention of Helicobacter pylori infection.

2. The Prior Arts

Helicobacter pylori are strongly associated with chronic active type B gastritis, peptic ulcers, gastric cancer, and gastric mucosa associated lymphoid tissue lymphoma. A 1-week combination therapy of a proton pump inhibitor and antibiotics is used as the treatment of choice for Helicobacter pylori infection currently. However, increasing resistance in Helicobacter pylori strains resistant to some of the abovementioned agents lead to eradication failure in some patients. Following failure of the initial treatment, 2nd-line therapies, including alternative triple and quadruple regimens, have been recommended.

The initial step in Helicobacter pylori infection is the penetration and adherence of the bacterium to mucin and gastric epithelial cells through several different adhesion molecules. Anti-adhesive therapy using 3′-sialyllactose has been shown to prevent the binding of Helicobacter pylori to various human gastrointestinal epithelial cells in vitro and to decrease Helicobacter pylori colonization in rhesus monkeys without side effects. After adhering to the gastric mucosa, Helicobacter pylori causes gastric epithelial cell damage and atrophy via oxidative stress and the type I apoptotic or type II autophagic programmed cell death-related pathway.

Catechins belong to well-characterized flavanol group of polyphenols that appear predominantly in tea. Catechin family is the most important tea polyphenols and takes up 75-80% of the polyphenol. There are four major derivatives of catechin: epicatechin (EC), epigallocatechin (EGC), epicatechin gallate (ECG), and epigallocatechin gallate (EGCG). Catechin and its derivative EGCG have antioxidative, antiinflammatory, antiapoptotic, and cancer prevention activities. Moreover, catechin and EGCG have antibacterial activity against various food-borne pathogenic bacteria and against Helicobacter pylori by inhibiting Helicobacter pylori urease, and vacuolating cytotoxin A activity.

Antibiotic treatment is effective most of the time in patients infected with Helicobacter pylori, but sometimes is ineffective because of antibiotic resistance or poor compliance. An alternative non-antibiotic agent or mixture with preventive and therapeutic effects is urgently required to treat Helicobacter pylori infection.

Some bacteria such as Lactobacillus or Bifidobacterium suppress the growth of Helicobacter pylori. However, clinical studies demonstrated that the probiotic treatment could not eradicate Helicobacter pylori completely. In addition, though vaccination was shown to be beneficial for the preventive and therapeutic treatment of Helicobacter pylori infection, it failed to eradicate Helicobacter pylori infection completely in infected mice though the bacterial load of Helicobacter pylori was lowered.

There are some of the non-antibiotic agents showed Helicobacter pylori inhibition activity. For example, adhesion receptor antagonists such as 3′-sialyllactose and antioxidants such as tea catechins, have shown reliable inhibitory effect on Helicobacter pylori infection in vitro. However, they fail to effectively control infection in animal models in vivo when each is used alone.

Previous studies on the effects of catechins or sialic acid treatment were the results of individual use. Effects of combined catechins/sialic acid treatment for patients with Helicobacter pylori infection have not yet been determined.

### SUMMARY OF THE INVENTION

Due to the limitation of current medication and treatment for increasing antibiotic resistance in patients with Helicobacter pylori infection, there is an urgent need to provide a composition or a combination for both prevention and treatment of Helicobacter pylori infection.

An objective of the present invention is to provide a composition for the prevention of Helicobacter pylori infection, comprising at least 128 mg/L catechin and its derivatives; at least 32 mg/L sialic acid; and water. The weight percentage of each component is based on the total weight of the composition.

Another objective of the present invention is to provide a composition for the treatment of Helicobacter pylori infection, which comprises from 128 mg/L to 640 mg/L catechin and its derivatives; from 32 mg/L to 160 mg/L sialic acid; and water. The Helicobacter pylori infection can be interfered or eradicated by the composition. That is to inhibit the interaction of Helicobacter pylori and gastric epithelial cells, at the same time to kill the bacteria but not affect the gastric epithelial cells.

The technology used in the present invention to solve the problems of Helicobacter pylori infection is to combine compositions including the anti-adhesion components, anti-bacteria Helicobacter pylori and antioxidant components such as sialic acid and green tea extract catechin and its derivatives to generate the protective ability against the damages in stomach caused by Helicobacter pylori. The major functions include: reducing the production of O₂.⁻, H₂O₂ and NO; decreasing the expression of inducible nitric oxide synthase (iNOS), increasing antioxidative activities through two-way regulation of autophagy and apoptosis in cells. The combination of catechins/sialic acid treatment can up-regulate autophagy, and decrease the apoptotic damage of stomach caused by Helicobacter pylori. The series in vivo and ex vivo studies have proved the effect of combined catechins/sialic acid for prevention and treatment of Helicobacter pylori infection.

The composition of the present invention can effectively decrease the gastric epithelial cell damage and lessen the gastric inflammation, which results in 100% prevention and 60% eradication in treatment of Helicobacter pylori infection. These two components occur in natural foods, can be obtained easily from intake of sialic acids and catechins abundant foods to prevent or treat the Helicobacter pylori related symptoms.

One of the components in the composition of the present invention is catechin and its derivatives. Catechin derivatives is selected from the group consisting of epigallocatechin gallate (EGCG), epicatechin gallate (ECG), gallocatechin gallate (GCG), epicatechin (EC), epigallocatechin (EGC), gallocatechin (GC), and any combination thereof.

The term ‘catechins’ in the present invention is consisting of catechin and above catechin derivatives.

The term ‘sialic acid’ in the present invention is a 9-carbon monosaccharide found in nature, formally called as N-Acetylneuraminic acid. It has a negative charge that causes the slippery texture of saliva.

The present invention is further explained in the following embodiment illustration and examples. Those examples below should not, however, be considered to limit the scope of the invention. It is contemplated that modifications will readily occur to those skilled in the art, which modifications will be within the spirit of the invention and the scope of the appended claims.

### BRIEF DESCRIPTION OF THE DRAWINGS

FIGS. 1A to 1D show changes of O₂⁻, H₂O₂, NO and iNOS in AGS cells at 4 h after infection with Helicobacter pylori (HP) or un-infected after the use of catechins (C) and sialic acid (S).

FIGS. 2A to 2B show apoptosis related protein levels in AGS cells at 4 h after infection with Helicobacter pylori (HP) or un-infected after the use of catechins (C) and sialic acid (S).

FIG. 3 shows autophagy related protein Beclin-1 levels in AGS cells at 4 h after infection with Helicobacter pylori (HP) or un-infected after the use of catechins (C) and sialic acid (S).

FIGS. 4A to 4L show morphology, apoptosis, and autophagy in AGS cells at 4 h after infection with Helicobacter pylori (HP) or un-infected after the use of catechins (C) and sialic acid (S).

FIGS. 5A to 5N show pathological analysis of gastric tissue in Helicobacter pylori (HP) infected or un-infected BALB/c mice.

FIG. 6 shows the Helicobacter pylori eradication rates with different dosages of catechins and sialic acid in infected BALB/c mice.

### DETAILED DESCRIPTION OF THE PREFERRED EMBODIMENT

The Score test and binomial test were used to test the statistical significant differences in Helicobacter pylori inhibitory effects of combined catechins/sialic acid treatment between the groups. One-way analysis of variance (ANOVA) and Duncan's Multiple Range test were used to examine the statistical significances among groups in the cell culture system. Kruskal-Wallis test, one-way ANOVA and Dunnett's multiple comparison with control method were used to examine differences among groups in the animal model. Simple logistic regression was applied to reveal the dosage effect on eradication rate. Differences with a P-value of 0.05 were considered significant.

Without intent to limit the scope of the present invention, the examples listed below are merely illustrative for convenience. All the weights, amounts or percentages are based on total weights or total amounts.

### Bacterial Strains and Drugs.

Standard strain ATCC 43504 and 20 clinical isolates (TA1-TA20) of Helicobacter pylori were used in the embodiments. The clinical isolates were obtained from gastric biopsy specimens from patients with gastritis and peptic ulcer after receiving the informed consents.

Decaffeinated green tea extracts purchased from Vigour Biochemistry consisted of 328 mg/g of epigallocatechin gallate (EGCG), 152 mg/g of epicatechin gallate ECG), 148 mg/g of gallocatechin gallate (GCG), 132 mg/g of epicatechin (EC), 108 mg/g of epigallocatechin (EGC), 104 mg/g of gallocatechin (GC), and 44 mg/g of catechin Sialic acid was obtained from Sigma.

### Example 1

### In Vitro Inhibitory Effects Toward Helicobacter pylori of Individual Catechins and Sialic Acid

The Helicobacter pylori inhibitory effects of catechins and sialic acid were tested in vitro. Twenty Helicobacter pylori strains to be tested were stored at −80° C. and recovered at 37° C. for 3 day under microaerophilic conditions (5% O₂, 10% CO₂, 85% N₂), then resuspended in 10 ml of Brucella broth for 24 h until an optical density at 450 nm of 0.5 units (corresponding to a concentration of 10⁹ CFU (colony-forming units)/L) was reached respectively. The minimal inhibitory concentrations (MIC) of catechins and sialic acid were determined by the agar dilution method. The effect of catechin and sialic acid combination was determined by the checkerboard method and evaluated using the fractional inhibitory concentration (FIC) index.

Referring to Table 1, the Helicobacter pylori inhibitory effects of catechins and sialic acid were shown in vitro. The MIC was determined as described previously. The MIC of the catechins for 90% of isolates (MIC₉₀) was 256 mg/L (Table 1). Sialic acid alone did not show any inhibitory activity against Helicobacter pylori.

TABLE 1
In vitro inhibitory effects toward 20 Helicobacter pylori strains of
catechins or sialic acid

| | MIC (mg/l) | | |
|---|---|---|---|
| Drug | Range | For 50% of strains | For 90% of strains |
| Catechins | 32-1024 | 128 | 256 |
| Sialic acid | >4000 | >4000 | >4000 |

* These clinical isolates consisted of 10 strains resistant to both metronidazole and clarithromycin, and 10 strains sensitive to both antibiotics.

### Example 2

### In Vitro Inhibitory Effects Toward Helicobacter pylori of Combined Catechins and Sialic Acid

Inhibitory effects toward twenty Helicobacter pylori strains were similarly tested with combined catechins and sialic acid in vitro. The results are shown in Table 2. The FIC was determined as described previously. All clinical isolates were susceptible to the combination of catechins and sialic acid, which had either an additive or a synergistic effect (Table 2). These data show that sialic acid enhanced the antibacterial activity of catechins. The checkerboard study (data not shown) demonstrated that the combination of 128 mg/L catechins and 32 mg/L sialic acid completely inhibited the growth of all clinical isolates tested in vitro. Antibiotic-sensitive and -resistant isolates did not differ in susceptibility to the combination of catechins and sialic acid.

TABLE 2

| | | | Additive | | |
|---|---|---|---|---|---|
| | Strains | Synergestic | 0.5 < | Indifferent | Antagonistic |
| Isolates | (n) | FIC ≦ 0.5 | FIC ≦ 1 | 1 < FIC ≦ 2 | FIC ≧ 2 |
| S | 10 | 4 | 6 | 0 | 0 |
| R | 10 | 3 | 7 | 0 | 0 |

* S, antibiotic-sensitive isolate;
R, antibiotic-resistant isolate to both metronidazole and clarithromycin.
Significant differences were shown between the groups with FIC ≦ 1 and FIC > 1, P < 0.01.

### Example 3

### The Oxidative Stress of Helicobacter pylori Infected Human Gastric Cancer Cells (AGS) Was Decreased after Catechins and Sialic Acid Treatment

### 1. Cell Culture

A cytotoxin-associated gene A-/vacuolating cytotoxin A-positive strain of Helicobacter pylori (TA1) was recovered from frozen stock by seeding on Columbia agar plate containing 5% sheep blood at 37° C. for 3 day under microaerophilic conditions. The human gastric cancer cell line ATCC CRL 1739 (AGS cells) was cultured in RPMI 1640 medium (Invitrogen) supplemented with 10% fetal bovine serum. For coculture of Helicobacter pylori and AGS cells, the bacteria were resuspended in PBS to 1.0 units at OD 450 nm, corresponding to a bacterial concentration of 2×10¹¹ CFU/L, and added to wells containing Helicobacter pylori and AGS cells to a ratio of 100:1. They were then cocultured for 4 h in the absence or presence of 128 mg/L of catechins and/or 32 mg/L of sialic acid.

### 2. Oxidative Stress Measurement.

The nitric oxide (NO) concentration was measured using an NO chemiluminescent probe and a Chemiluminescence Analyzing System (CLD-110, Tohoku Electronic). For measurement of O₂⁻ and hydrogen peroxide (H₂O₂), a 0.2 ml culture sample and 0.5 ml of 0.1 mmol/L lucigenin or 0.2 mmol/L luminol in PBS (pH 7.4) was used for chemiluminescence assay. The assay was performed in triplicate and was expressed as the chemiluminescence count per 10 s.

3. Quantification on Expression of iNOS.

Western blot analysis was employed for the quantification of iNOS expression. Proteins were extracted from the cells after 4-h treatment and electrophoresed on 10% SDS-PAGE, and transferred into polyvinylidene difluoride membranes using a semidry transfer system (Hoeffer Phamacia Biotech). The membranes were blocked for 2 h at room temperature in PBS containing 5% milk, and incubated with buffers containing antibodies against inducible NO synthase (iNOS) (Chemicon), Bax, Bcl-2, caspase 3, poly-(ADP-ribose)-polymerase (all from Cell Signaling Technology), or Beclin-1 (BD Biosciences) for 1 h at room temperature. The membranes were then washed 3 times and incubated for 1 h at room temperature with blocking buffer containing horseradish peroxidase conjugated rabbit anti-IgG antibody (Pierce). The signals were detected by enhanced chemical luminescence (Amersham Biosciences) and exposure to X-ray film.

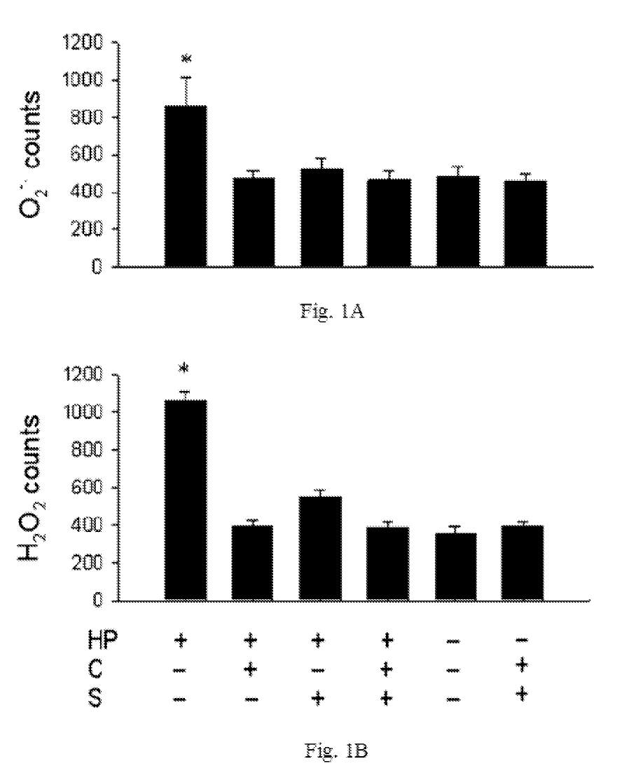

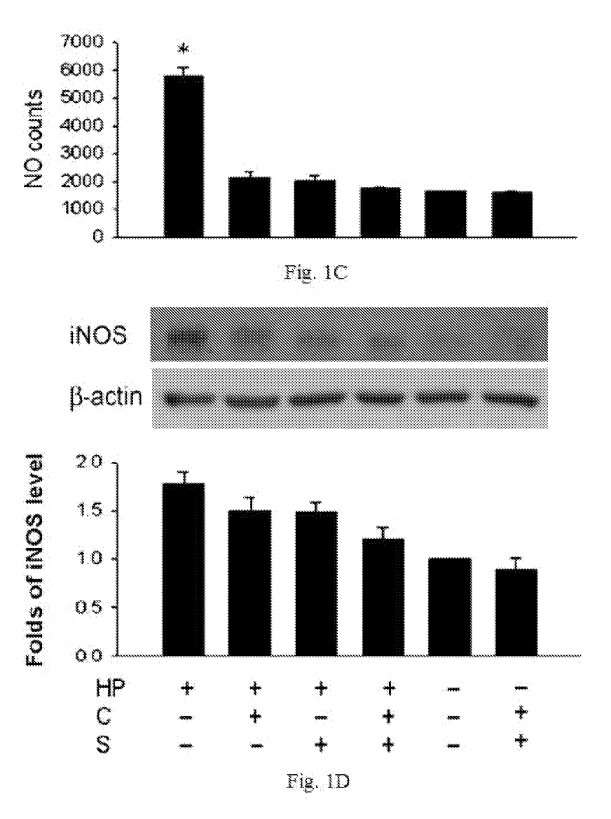

FIGS. 1A-1D showed the production of O₂⁻, H₂O₂, NO and expression of iNOS in AGS cell cultures after Helicobacter pylori infection for 4 h in the presence of catechins and sialic acid. The results from Western blot were also included in FIG. 1D. Helicobacter pylori infection resulted in DNA damage, considerate production of reactive oxygen species (ROS) and increase of iNOS from previous studies. The antioxidant effects can be evaluated from this experiment. The production of O₂⁻, H₂O₂, NO and expression of iNOS were increased after 1 h of Helicobacter pylori infection and persisted until 4 h after infection. These effects were significantly suppressed by the presence of catechins and sialic acid. Therefore, antioxidant activity was enhanced in cells after the addition of catechins and sialic acid.

### Example 4

### Apoptosis of Helicobacter pylori Infected Human Gastric Cancer Cells (AGS) was Decreased after Catechins and Sialic Acid Treatment

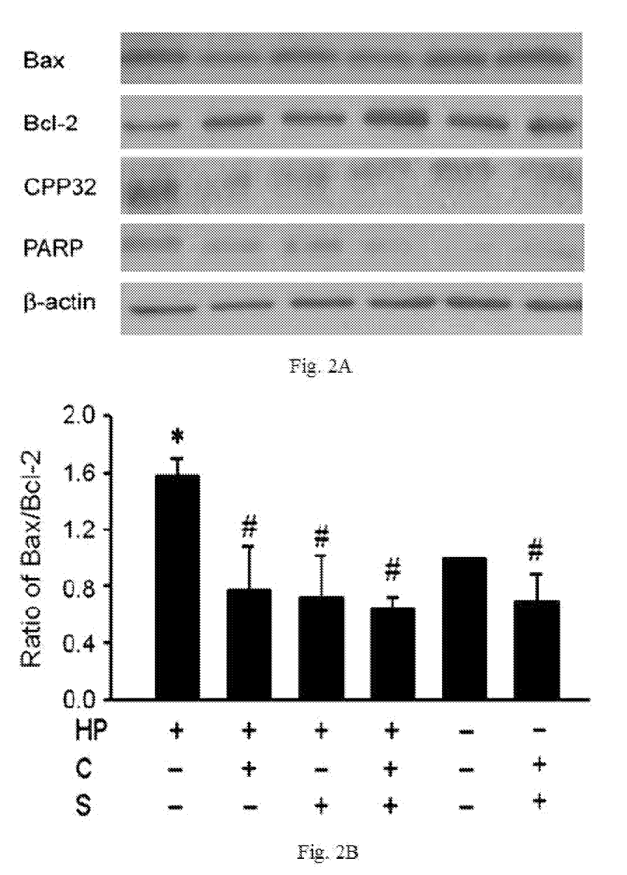

FIGS. 2A-2B showed the expression levels of apoptosis related proteins in AGS cell cultures after Helicobacter pylori (HP) infection for 4 h in the presence of catechins (C) and sialic acid (S). The condition of gel electrophoresis was the same as abovementioned. Bax, Bcl-2, CPP32 and PARP were all related to cell apoptosis. The ratio of Bax and Bcl-2 were used as indexes. Helicobacter pylori infection increased expression of Bax and decreased expression of Bcl-2 in AGS cells, indicating that the increased Bax:Bcl-2 ratio enhanced apoptosis in Helicobacter pylori-infected AGS cells (FIG. 2). Treatment of catechins and sialic acid suppressed Bax expression and increased Bcl-2 expression, indicating that catechins and sialic acid could reduce the apoptotic effect of Helicobacter pylori infection. Both the expression of CPP32 (caspase 3) and the expression of PARP (poly-(ADP-ribose)-polymerase) were also significantly inhibited by the treatment of catechins and sialic acid in the Helicobacter pylori infected AGS cell culture. Therefore, treatment of catechins and sialic acid decreased the apoptosis of AGS cells.

### Example 5

### The Autophagy of Helicobacter pylori Infected Human Gastric Cancer Cells (AGS) was Increased after Catechins and Sialic Acid Treatment

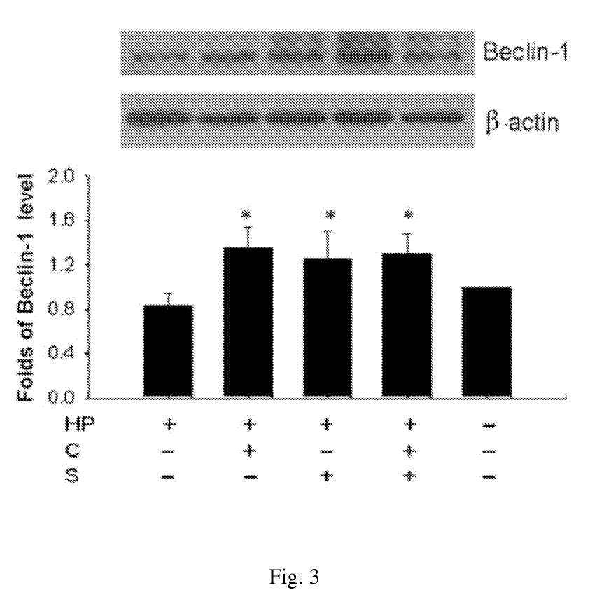

FIG. 3 showed the expression levels of autophagy related protein Beclin-1 in AGS cell cultures after Helicobacter pylori (HP) infection for 4 h in the presence of catechins (C) and sialic acid (S). The condition of gel electrophoresis was the same as abovementioned. Beclin-1 is thought to interact with Bcl-2 to facilitate autophagic cell death. As indicated in FIG. 3, Helicobacter pylori infection decreased the expression of the Beclin-1, but treatment of catechins and sialic acid stimulated the production of Beclin-1 and enhanced Beclin-1-dependent autophagy.

The morphology changes of Helicobacter pylori (HP) infected-AGS cells after treatment of catechins and sialic acid were observed under a fluorescent microscope. Autophagic vacuoles were labeled in triplicate with 0.05 mmol/L monodansylcadaverine (MDC). After labeling, the cells were washed 4 times with PBS and immediately fixed with 4% paraformaldehyde and observed under a fluorescence microscope (Leica model DMRD). Helicobacter pylori-induced AGS cell apoptosis was assayed in triplicate using the terminal deoxynucleotidyl transferase-mediated nick-end labeling method.

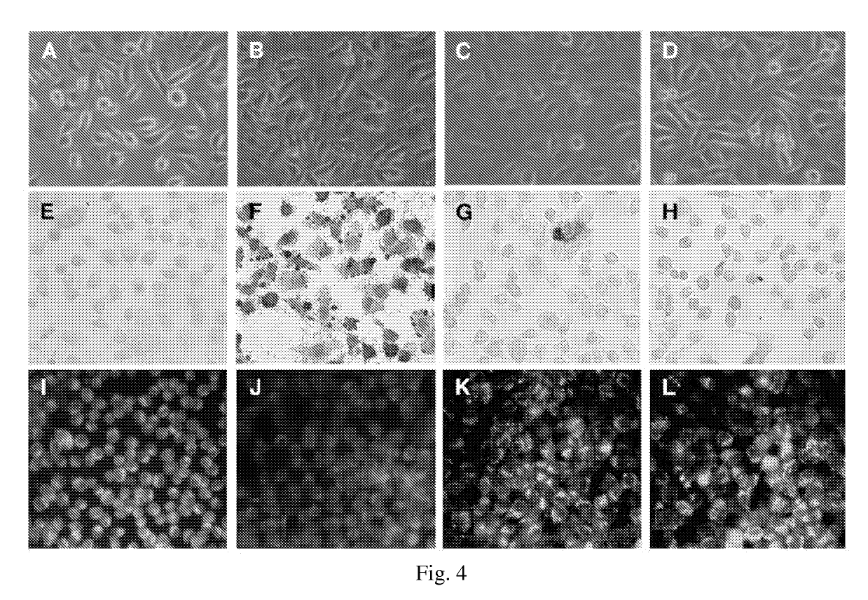

FIGS. 4A-4L showed morphologic changes, apoptosis formation, and inhibition of autophagy on AGS cells after 4 h of Helicobacter pylori and the treatment of catechins and sialic acid. FIGS. 4A-4D were the phase contrast images of cells without staining, FIGS. 4E-4H were the cells stained with the terminal deoxynucleotidyl transferase-mediated nick-end labeling method, and FIGS. 4I-4L showed the cells stained with 0.05 mM of MDC.

FIGS. 4A, 4E and 4I showed the morphology of intact cells, no cell apoptosis, and minor cell autophagy respectively, while FIGS. 4B, 4F and 4J showed distinct morphologic change, apoptosis and loss of autophagy after Helicobacter pylori infection. FIGS. 4C, 4G and 4K showed cells treated with catechins and sialic acid to preserve the cell morphology, inhibit apoptosis, and maintain autophagy respectively. And FIGS. 4D, 4H, and 4L showed no harmful effects to cells after the catechins and sialic acid treatment in control cells.

### Example 6

### Catechins and Sialic Acid Treatment in Animal Models

Five-wk-old male specific pathogen-free BALB/c mice were obtained from the National Laboratory Animal Center, Taiwan, and housed at the Experimental Animal Center, National Taiwan University at a constant temperature. Mice consumed food (20.5% of protein, 18.5% of fat, 53% of carbohydrate, 2.7% of fiber, and 4.8% of mineral) and water ad libitum. All surgical and experimental procedures were approved by the Institutional Animal Care and Use Committee of the National Taiwan University College of Medicine and were in accordance with the guidelines of the National Science Council of Taiwan.

Forty mice were divided into 4 groups of 10 mice each. The Helicobacter pylori strain TA1 was used to infect mice. The recovered bacterial colonies were transferred to Brucella broth supplemented with 5% fetal bovine serum, 1% IsoVitaleX, and antibiotics and maintained for 48 h, and adjusted to 10¹¹ CFU/L. Mice in treatment groups were orally administered 2 times on successive days with 0.5 ml of bacterial suspension, while control mice received distilled water only. The mice in the pretreatment group were fed with 0.5 ml of distilled water containing 128 mg/L of catechins and 32 mg/L of sialic acid 72 h before Helicobacter pylori inoculation, then had free access to drinking water containing 1% glucose and a mixture of 128 mg/L of catechins and 32 mg/L sialic acid for 3 d. Mice in the post-treatment group were post-treated with 0.5 ml of distilled water containing 128 mg/L of catechins and 32 mg/L of sialic acid at 2 wk after Helicobacter pylori inoculation, then had free access to drinking sialic acid solution for 5 d. The infected controls received 1% glucose water orally for 3 d before to 5 d after infection. All procedures other than those described above were the same in all 4 study groups. Four wk after Helicobacter pylori inoculation, the stomachs of mice were removed and longitudinally divided into 2 equal parts for histological and microbiological examination.

Histological staining was carried out by in situ staining of 3-nitrotyrosine (3-NT) and 4-hydroxynonenal (4-HNE) in Helicobacter pylori-infected gastric tissues, which was immunostaining in paraffin-embedded sections. The sections were incubated overnight at 4° C. 4 with rabbit anti-nitrotyrosine IgG antibodies (NITT12-A) or rabbit anti-HNE antibodies HNE11-S (both were from Alpha Diagnostic) diluted 1:50 in PBS, then stained by an avidin-biotinylated horseradish-peroxidase procedure using a commercially available kit (ABC Elite, Vector Laboratories). The signal was visualized by incubating the sections with liquid diaminobenzidine tetrahydrochloride. Hematoxylin was used to counter-stain the sections.

Helicobacter pylori could be identified after 3-5 d culture after the abovementioned histological staining and microbiological test. The CFU of Helicobacter pylori was counted after culturing. Gastritis was graded by the pathologist. In addition, infection of Helicobacter pylori was confirmed by PCR.

The effects of catechins and sialic acid on Helicobacter pylori infected mice was shown in Table 3. All mice in the inoculated control group were successfully infected with obvious edema and hemorrhage in gastric mucosa.

TABLE 3

| | | | Macroscopic | Bacterial count | |
|---|---|---|---|---|---|
| Groups | Mice (n) | HP infection (%) | damage (%) | 10³ CFU/g | Gastritis score |
| Uninfected | 10 | 0 (0)^{#} | 0 (0)^{#} | 0^{#} | 0.3 ± 0.5^{#} |
| Pretreated | 10 | 0 (0)^{#} | 0 (0)^{#} | 0^{#} | 0.3 ± 0.5^{#} |
| Post-treated | 10 | 8 (80)* | 6 (60)* | 1.0 ± 0.8^{#,} * | 0.8 ± 0.6^{#} |
| Infected | 10 | 10 (100)* | 9 (90)* | 180 ± 90* | 2.0 ± 0.7* |

Values are mean ± SD,
n = 10 or n (%).
*Different from the uninfected control in that column;
^{#}different from the infected control in that column,
P < 0.01.

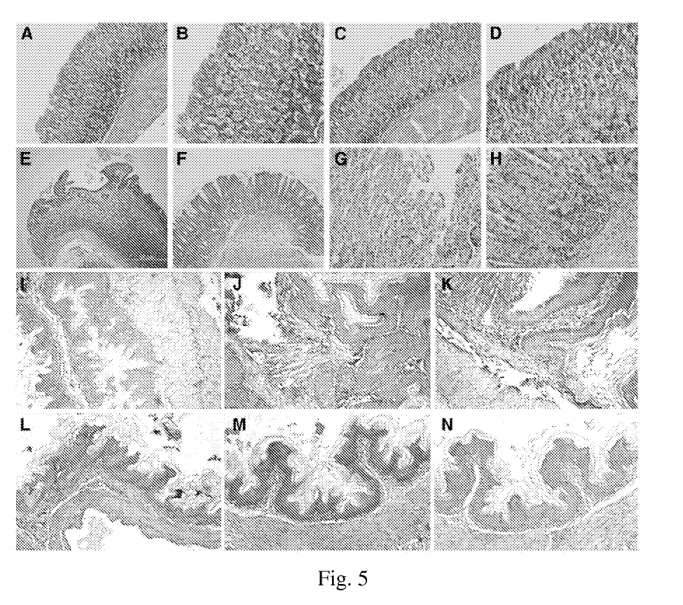

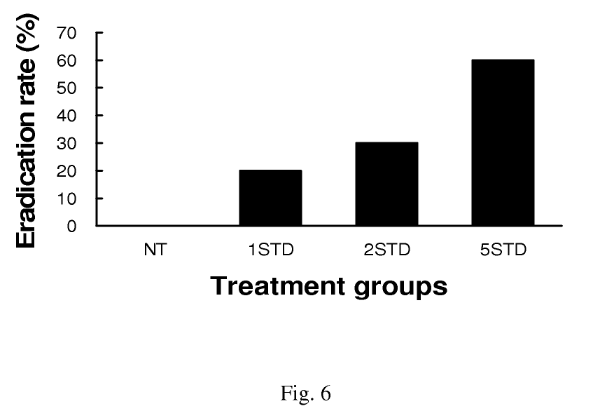

Referring to FIGS. 5A-5N, pathological analysis of gastric tissue in each group of BALB/c mice was shown. Microscopically, prominent gastritis with infiltration of many mononuclear cells and neutrophils was observed (FIGS. 5F-H). FIG. 5F showed gastric mucosa of an untreated Helicobacter pylori-infected mouse with inflammatory changes in the mucosa and superficial submucosa (H&E stain, 100×). FIG. 5G showed mucosal destruction in the gastric mucosa of an untreated Helicobacter pylori-infected mouse (H&E stain, 400×). FIG. 5H showed multiple neutrophilic aggregation and micro-abscess formation in the lower portion of the mucosa of an untreated Helicobacter pylori-infected mouse (H&E stain, 400×). The mean gastritis score was 2.0 (Table 3).

In the pretreated group (mice pretreated with catechins and sialic acid), none of the mice were infected with Helicobacter pylori and had no gross mucosal injury. There was only minimal histological change microscopically (FIGS. 5C-5D). The mean gastritis score was 0.3, the same as in un-infected mice (FIGS. 5A-5B).

The erosive lesion in mucosa was starting to be repaired in the post-treated group after the treatment of catechins and sialic acid (H&E stain, 100×; FIG. 5E). 20% of the mice were cleared of Helicobacter pylori infection. Although most Helicobacter pylori uneradicated mice in this group had gross mucosal injury, the average score of 0.8 for microscopic gastritis was lower (P<0.01) than that in the infected control group. The mean gastritis score of uninfected, pretreated, and post-treated groups were all significantly differed from the infected control group (all P<0.01).

Referring to FIGS. 5I-5N, 3-NT staining (in brown color) in the proximal part of the gastric mucosa was shown in FIGS. 5I-5K, while 4-HNE staining (in brown color) for FIGS. 5L-5N. FIG. 5I and FIG. 5L showed the results of the control group, FIGS. 5J and 5M, the Helicobacter pylori-infected group, and FIGS. 5K and 5N, the catechins and sialic acid-pretreated Helicobacter pylori-infected group. The un-infected control group (FIGS. 5I and 5L) showed no oxidation in blue, the infected group displayed obvious brown (FIGS. 5J and 5M), and the pretreated group also revealed blue (FIGS. 5K and 5N) as the un-infected group. Briefly, the accumulation of 3-NT and 4-HNE compounds in the proximal part of the gastric mucosa of infected mice was revealed, and the amounts accumulated were higher than catechins and sialic acid-pretreated Helicobacter pylori-infected group.

Inhibitory Effects of Helicobacter pylori was in a Dose-Dependent Manner in Post-Treatment of Catechins and Sialic Acid

Different amounts of catechins and sialic acid were applied to test the inhibitory effects of Helicobacter pylori. 60 mice were divided into 3 groups for 3 dosages: one standard dose, 2-fold dose and 5-fold dose. Another 10 mice were used for none-treatment (NT) control group. The eradication rates were evaluated after 4 weeks of treatment.

The Helicobacter pylori eradication rates were 0% in the none-treatment group, 20% in the one standard dose group (128 mg/L catechins and 32 mg/L sialic acid), 30% in the 2-fold dose group, and 60% in the 5-fold dose group (FIG. 6). The amount of dosage was positively related to the eradication rate with significance (P<0.01), with odds ratio of 1.695 for every fold of standard dose added.

In summary, the composition of catechins and sialic acid in the present invention completely prevented Helicobacter pylori infection and eradicated up to 60% of Helicobacter pylori infection in a dose dependent manner. This composition is shown to have significant effect in Helicobacter pylori inhibition. These above examples should not, however, be considered to limit the scope of the invention.

What is claimed is:

1. A composition for the prevention of Helicobacter pylori infection, comprising at least 0.128 wt % c catechin and its derivatives; at least 0.032 wt % c sialic acid; and water.

2. The composition according to claim 1, wherein the catechin derivatives is selected from the group consisting of epigallocatechin gallate (EGCG), epicatechin gallate (ECG), gallocatechin gallate (GCG), epicatechin (EC), epigallocatechin (EGC), gallocatechin (GC), and any combination thereof.

3. The composition according to claim 1, wherein the catechin is extracted from a tea extract.

4. The composition according to claim 3, wherein the tea extract is a decaffeinated green tea extract.

5. The composition according to claim 1, wherein the composition is applied in a stomach.

6. The composition according to claim 1, wherein the composition is for oral administration.

7. The composition according to claim 1, wherein the composition increases the antioxidant activity of a gastric mucosal cell through reducing the production of O₂.⁻, H₂O₂ and NO, and decreasing the expression of inducible nitric oxide synthase (iNOS) in the normal cell.

8. A composition for the treatment of Helicobacter pylori infection, which comprises from 128 mg/L to 640 mg/L catechin or its derivatives; from 32 mg/L to 160 mg/L sialic acid; and water.

9. The composition according to claim 8, wherein the catechin derivatives is selected from the group consisting of epigallocatechin gallate (EGCG), epicatechin gallate (ECG), gallocatechin gallate (GCG), epicatechin (EC), epigallocatechin (EGC), gallocatechin (GC), and any combination thereof.

10. The composition according to claim 8, wherein the catechin is extracted from a tea extract.

11. The composition according to claim 10, wherein the tea extract is a decaffeinated green tea extract.

12. The composition according to claim 8, wherein the composition is applied in a gastric disease.

13. The composition according to claim 12, wherein the gastric disease is chronic active gastritis, peptic ulcers, gastric cancer, or gastric mucosa-associated lymphoid tissue lymphoma.

14. The composition according to claim 8, wherein the composition is for oral administration.

15. The composition according to claim 8, wherein the composition increases the antioxidant activity of an infected cell through reducing the production of O₂⁻, H₂O₂ and NO, and decreasing the expression of inducible nitric oxide synthase (iNOS) in the infected cell.

16. The composition according to claim 8, wherein the composition reduces the damage of a stomach caused by Helicobacter pylori through up-regulation of autophagy and down-regulation of apoptosis.
